(19) United States
(12) Patent Application Publication
(10) Pub. No.: US 2024/0123431 A1
(43) Pub. Date: Apr. 18, 2024

(54) NOVEL SUPRAMOLECULAR SELF-ASSEMBLY, CARBON NITRIDE AND PHOTOCATALYST USING SAME, AND MANUFACTURING METHOD THEREFOR

(71) Applicant: University of Ulsan Foundation for Industry Cooperation, Ulsan (KR)

(72) Inventors: Byeong Kyu LEE, Ulsan (KR); Milad JOURSHABANI, Ulsan (KR)

(73) Assignee: University of Ulsan Foundation for Industry Cooperation, Ulsan (KR)

(21) Appl. No.: 18/285,669

(22) PCT Filed: Jul. 5, 2021
(86) PCT No.: PCT/KR2021/008482

§ 371 Date: Oct. 5, 2023

(30) Foreign Application Priority Data
Apr. 7, 2021 (KR) 10-2021-0045141

(51) Int. Cl. B01J 23/28; B01J 23/22; B01J 23/30; B01J 27/24; B01J 35/39; B01J 35/61; B01J 35/63; B01J 35/64; B01J 37/08

(52) U.S. Cl. CPC B01J 23/28; B01J 23/22; B01J 23/30; B01J 27/24; B01J 35/39; B01J 35/615; B01J 35/635; B01J 35/651; B01J 37/08

(57) ABSTRACT

The present invention relates to a novel supramolecular self-assembly, a carbon nitride and a photocatalyst using same, and a manufacturing method therefor. The present invention can provide, by using a supramolecular self-assembly, a carbon nitride having a high N—C═N bonding ratio, a photocatalyst having excellent photocatalytic activity under visible light, and a manufacturing method therefor, the supramolecular self-assembly comprising: a plurality of complex units formed by hydrogen bonding two or more nitrogen-containing compounds to each other; and linker units connecting the plurality of complex units by hydrogen bonds, wherein the nitrogen-containing compounds and the linker units are each independently a —NH group and capable of hydrogen bonding with the —NH group, and the supramolecular self-assembly contains one or more heteroatoms selected from the group consisting of N, S, and O.

### TECHNICAL FIELD

The present invention relates to a novel supramolecular self-assembly, a carbon nitride and a photocatalyst using the same, and a method of manufacturing the supramolecular self-assembly.

### BACKGROUND ART

Water shortage and environmental problems caused by a rapidly-growing population and urbanization have become major concerns around the world. Reuse of waste water is one of the useful ways to solve these water shortage and environmental problems and provide water sources for agricultural and industrial activities. A major concern in wastewater reuse is to remove residual organic compounds, which are expensive to dispose of. In particular, stable aromatic organic compounds such as pharmaceuticals are considered major contaminants of waste water. For example, tetracycline, which is used as a drug for humans and animals, is frequently detected in surface water, and various organic dyes have been used in various fields such as paper, textile, cosmetics, printing, and leather industries. These organic compounds are difficult to degrade biologically and are potentially toxic.

Advanced oxidative processes (AOPs) have recently received increasing attention as a promising method of removing recalcitrant organic compounds from water. In general, AOPs are known to generate active species (radicals) such as ·OH or ·O₂⁻, which are highly reactive with aquatic contaminants and microorganisms.

Among various AOPs, photocatalysts may remove organic contaminants by utilizing solar energy. When a photocatalyst absorbs photons with energy exceeding the bandgap, excited electrons may be obtained from a valence band to a conduction band, and electron holes are formed by the excited electrons. The excited electrons are captured by O₂ and H₂O molecules, thereby generating the above-described radicals. The generated radicals degrade organic contaminants through a series of oxidation/reduction reactions.

As one of the semiconductor photocatalysts, titanium dioxide (TiO₂) has been widely used for wastewater disposal, and TiO₂ has been reported to have properties such as high photocatalytic activity, solubility, non-toxicity, and high stability. However, TiO₂ has limited photocatalytic activity due to its wide bandgap such as 3.0 eV (rutile) and 3.2 eV (anatase). It can only absorb 3 to 5% of sunlight, so its application under visible light is inevitably limited (Patent Document 1). Also, post-separation of TiO₂ particles suspended in a liquid phase is difficult due to the presence of fine particles in a slurry state, and the technology for supporting a photocatalyst on a porous reactive surface requires high costs for use in wastewater disposal (Patent Document 2).

### DISCLOSURE

### Technical Problem

The present invention is directed to providing a novel supramolecular self-assembly, a carbon nitride having a high N—C═N bond ratio using the same, a photocatalyst having excellent photocatalytic activity under visible light, and a method of manufacturing the supramolecular self-assembly.

### Technical Solution

According to one aspect of the present invention, there is provided a supramolecular self-assembly which includes a plurality of complex units formed by hydrogen bonding of two or more nitrogen-containing compounds to each other; and a linker unit configured to connect the plurality of complex units via a hydrogen bond, wherein the nitrogen-containing compounds and the linker unit each independently include an —NH group and one or more heteroatoms capable of hydrogen bonding with the —NH group and selected from the group consisting of N, S, and O.

According to one embodiment, at least one of the nitrogen-containing compounds may include S or O, which may be different from the heteroatoms included in the linker unit.

According to one embodiment, the nitrogen-containing compounds may include a first nitrogen-containing compound having an —NH group and N, and a second nitrogen-containing compound having an —NH group and O, and the linker may include a compound containing an —NH group and S.

According to one embodiment, the plurality of complex units may include a 1,3,5-triazine framework and a 1,3,5-triazinane framework.

According to one embodiment, the linker may include thiourea, a thiourea dimer, or a combination thereof.

According to one embodiment, the supramolecular self-assembly may show a peak at 2θ=10.8°±0.4°, 11.8°±0.4°, 28.1°±0.4°, or 33.2°±0.4°, as measured by X-ray diffraction using CuKα rays.

According to one embodiment, the supramolecular self-assembly may show a peak at 1,084±20 cm⁻¹, as measured by FT-IR.

According to another aspect of the present invention, there is provided a method of manufacturing a supramolecular self-assembly, the supramolecular self-assembly being manufactured by a hydrothermal reaction using a precursor, wherein the precursor includes a nitrogen-containing compound having an —NH group; and a compound capable of hydrogen bonding with the —NH group and having one or more heteroatoms selected from the group consisting of N, S, and O.

According to one embodiment, the precursor may include the following (a) to (c):
(a) a compound containing 2 to 6 nitrogen atoms;
(b) a compound containing 2 to 4 nitrogen atoms and one or more oxygen atoms; and
(c) a compound containing one or more nitrogen atoms and one or more sulfur atoms.

According to one embodiment, the molar ratio of the compound (a) or (b) to the compound (c) may be in a range of 1:0.2 to 1:2.

According to one embodiment, the hydrothermal reaction may be performed at 60° C. to 180° C. for 1 to 12 hours after the precursor is dissolved in a solvent.

According to still another aspect of the present invention, there is provided a carbon nitride including a heptazine framework,
wherein a peak representing C—C binding energy is present at 284.8±1 eV and a peak representing an N—C═N bond is present at 288.1±1 eV, as analyzed by C is X-ray photoelectron spectroscopy (XPS), and when it is assumed that the highest peak value observed at 284.8±1 eV is I₁, and the highest peak value observed at 288.1±1 eV is I₂, I₂/I₁ is greater than or equal to 2.

According to one embodiment, the carbon nitride may have a bandgap energy of 2.7 eV to 3.0 eV.

According to yet another aspect of the present invention, there is provided a method of manufacturing a carbon nitride, which includes: polycondensing and heat-treating the above-described supramolecular self-assembly to manufacture a carbon nitride.

According to one embodiment, the polycondensation may be performed at 500° C. to 600° C. for 2 hours to 5 hours.

According to one embodiment, the heat treatment may be performed at 450° C. to 550° C. for 1 to 5 hours.

According to yet another aspect of the present invention, there is provided a photocatalyst including the above-described carbon nitride and a metal oxide formed on a surface of the carbon nitride and/or inside the carbon nitride.

According to one embodiment, the metal oxide may include at least one selected from tungsten, vanadium, and molybdenum.

According to one embodiment, the photocatalyst may have a pore size of 30 nm or more, a pore volume of 0.3 cm³/g or more, and a BET specific surface area of 100 m²/g or more.

According to yet another aspect of the present invention, there is provided a method of manufacturing a photocatalyst, which includes:
polycondensing the above-described supramolecular self-assembly; and heat-treating the polycondensed self-assembly, wherein the polycondensing is performed by dispersing a metal-containing precursor and the self-assembly in a solvent to perform the polycondensation, or the heat-treating is performed by dispersing a metal-containing precursor and the polycondensed self-assembly in a solvent to perform the heat treatment.

### Advantageous Effects

The present invention can provide a carbon nitride having a high N—C═N and/or C—N═C bond ratio using a novel supramolecular self-assembly and can also provide a photocatalyst having excellent photocatalytic activity under visible light.

### DESCRIPTION OF DRAWINGS

FIG. 1 is a schematic diagram showing a photocatalyst synthesis method according to the present invention.

FIG. 2A is a conceptual diagram of a supramolecular self-assembly.

FIG. 2B is an image of the supramolecular self-assembly according to the molar ratio of precursors (melamine, cyanuric acid, thiourea).

FIG. 3 shows the X-ray diffraction (XRD) spectra (a) and Fourier transform infrared spectroscopy (FT-IR) spectra (b) of Preparation Example and Comparative Examples 1 to 3.

FIG. 4 is an enlarged diagram of a sulfur (S) region in the Fourier transform infrared spectroscopy (FT-IR) spectra (b) of Preparation Example and Comparative Examples 1 to 3.

FIG. 5 is a scanning electron microscope (SEM) image of Example 1 and Comparative Example 4.

FIG. 6 is a transmission electron microscope (TEM) image of Example 1.

FIG. 7 shows the Fourier transform infrared spectroscopy (FT-IR) spectra of Example 1 and Comparative Examples 4 and 5.

FIG. 8 shows the high-resolution X-ray photoelectron spectroscopy (XPS) spectra of C is (a) and N is (b) of Example 1 and Comparative Examples 4 and 5.

FIG. 9 shows the diffuse reflectance spectroscopy (DRS) spectra (a) and Tauc plots (b) of Example 1 and Comparative Examples 4 and 5.

FIG. 10 shows the X-ray diffraction (XRD) spectra of Example 2 and Comparative Examples 4, 6 and 7.

FIG. 11 is a transmission electron microscope (TEM) image of Example 2.

FIG. 12 shows the high-resolution X-ray photoelectron spectroscopy (XPS) spectra of C is (a) and N is (b) of Example 2 and Comparative Examples 4 and 6.

FIG. 13 shows the diffuse reflectance spectroscopy (DRS) spectrum (a) and Tauc plots (b) of Example 2 and Comparative Examples 4 and 6.

FIG. 14 is a graph showing the photocatalytic activities (a) and reaction rate constant plots (b) of Examples 1 to 4 and Comparative Examples 4 and 5 in the photocatalytic degradation of rhodamine B present at a concentration of 12 mg/L in an aqueous solution.

FIG. 15 is a graph showing the photocatalytic activities (a) and reaction rate constant plots (b) of Examples 1 to 4 and Comparative Examples 4 and 5 in the photocatalytic degradation of tetracycline at a concentration of 20 mg/L in an aqueous solution.

FIGS. 16A and 16B show liquid chromatography-mass spectrometry (LC-MS) chromatograms for tetracycline photolysis and related intermediates of Example 2.

### BEST MODE

The present invention may be modified in various ways and have various alternative forms, and thus specific embodiments thereof will be shown in the drawings and described in detail in the detailed description.

However, it should be understood that the present invention is not intended to limit the specific embodiments, but is intended to cover all modifications, equivalents, and alternatives falling within the spirit and scope of the present invention.

In the present invention, the terms “comprise,” “comprising,” “include,” “including,” “have,” and/or “having,” when used herein, specify the presence of stated features, integers, steps, operations, elements, components and/or groups thereof, but do not preclude the presence or addition of one or more other features, integers, steps, operations, elements, components and/or groups thereof.

Therefore, because the configurations described in the embodiments described in this specification are only the most preferred embodiments of the present invention and do not represent all the technical ideas of the present invention, it should be understood that there may be various equivalents and modifications that may replace them at the time of filing the present application.

Carbon nitride is a binary compound in which carbon and nitrogen are alternated to form covalent bonds. The carbon nitride of the present invention includes a solid phase in which sp²-hybrid carbon and nitrogen atoms are present in a heptazine unit, which leads to a π-conjugated electron structure, and a polymer material including the same, which is not standardized and has various sizes and structures.

Conventional bulk carbon nitride had a problem of low photocatalytic activity due to a low surface area and rapid recombination of excited electron-hole pairs by light. However, the present invention provides a more condensed carbon nitride through the polycondensation of a novel supramolecular self-assembly. Furthermore, the present invention may provide a photocatalyst having a wider light absorption range and excellent oxidation/reduction reactivity by forming a heterojunction between the carbon nitride and a metal.

The supramolecular self-assembly is a stable aggregate of molecules in which the molecules are gathered and bonded through intermolecular forces such as hydrogen bonds, ionic bonds, and van der Waals forces under equilibrium conditions. A starting material having hydrogen (H), nitrogen (N), sulfur (S), or oxygen (O) atoms forms multiple hydrogen bonds, and may become a new starting material for carbon nitrides having new physical properties.

The present invention provides a supramolecular self-assembly including a plurality of complex units formed by hydrogen bonding of two or more nitrogen-containing compounds to each other; and a linker unit configured to connect the plurality of complex units via a hydrogen bond.

The nitrogen-containing compounds and the linker unit may each independently include an —NH group and one or more heteroatoms capable of hydrogen bonding with the —NH group and selected from the group consisting of N, S, and O.

The nitrogen-containing compounds include a —NH group, and at least one of the nitrogen-containing compounds includes S or O, which may be different from the heteroatoms included in the linker unit.

Specifically, the nitrogen-containing compounds may include a first nitrogen-containing compound having an —NH group and N, and a second nitrogen-containing compound having an —NH group and O. Also, the nitrogen-containing compounds may contain a C—N single bond, a double bond, or a triple bond.

The plurality of complex units formed by hydrogen bonding of the two or more nitrogen-containing compounds to each other may include a 1,3,5-triazine framework or a heptazine framework. Also, the plurality of complex units may further include a 1,3,5-triazinane framework. The 1,3,5-triazine framework includes 1,3,5-triazine and derivatives of 1,3,5-triazine, and the heptazine framework includes heptazine and derivatives of heptazine, and the 1,3,5-triazineine framework includes 1,3,5-triazineine and derivatives of 1,3,5-triazineine. The derivative of 1,3,5-triazine may be 1,3,5-triazine-2,4,6-triamine or 1,3,5-triazine-2,4,6-triol.

Also, each of the complex units may be a melamine-cyanurate complex in which melamine and cyanurate are bonded via a hydrogen bond.

The complex units may have a two-dimensional structure formed on the same plane.

The linker unit may include one or more heteroatoms capable of hydrogen bonding with the —NH group of the plurality of complex units and selected from the group consisting of N, S, and O. Specifically, the linker may include a compound containing an —NH group and S. More specifically, the linker may include thiourea, a thiourea dimer, or a combination thereof.

The linker includes a heteroatom, particularly S, and thus may connect the plurality of complex units to each other via a hydrogen bond without any formation in the complex unit. More specifically, the linker connects a plurality of complex units having a two-dimensional structure to each other via a hydrogen bond so that the supramolecular self-assembly can form a three-dimensional structure. In this case, the supramolecular self-assembly formed in the three-dimensional structure may have a hexagonal pillar or hexagonal shape. The hexagonal pillar or hexagonal shape may have a length of 0.1 μm to 2 μm in a thickness direction and a length of 0.1 μm to 20 in a longitudinal direction.

The supramolecular self-assembly may show a peak at 2θ=10.8°±0.2°, 11.8°±0.2°, 21.9°±0.2°, 28.1°±0.2°, or 33.2°±0.2°, as measured by X-ray diffraction (XRD) using CuKα rays. Specifically, the supramolecular self-assembly may have a peak at 20=10.8°±0.2°, 11.8°±0.2°, 21.9°±0.2°, 28.1°±0.2°, or 33.2°±0.2°, as measured by X-ray diffraction (XRD) using CuKα rays. Such a peak may not be observed when each of the first nitrogen-containing compound, the second nitrogen-containing compound, and the linker are measured by X-ray diffraction. Therefore, it can be seen that the supramolecular self-assembly according to the present invention is a novel supramolecular self-assembly in which each of the first nitrogen-containing compound, the second nitrogen-containing compound, and the linker is newly oriented by a hydrogen bond.

Also, the supramolecular self-assembly may show a peak at 1,086±10 cm⁻¹, particularly a peak at 1,086±5 cm⁻¹, 1,086±1 cm⁻¹, or 1,086±0.5 cm⁻¹, as measured by Fourier transform infrared spectroscopy (FT-IR). It can be seen that the peak represents the C═S stretching vibration, indicating that the supramolecular self-assembly contains S.

In addition, the present invention may provide a method of manufacturing a supramolecular self-assembly, the supramolecular self-assembly being manufactured by a hydrothermal reaction using a precursor, wherein the precursor includes a nitrogen-containing compound having an —NH group; and a compound capable of hydrogen bonding with the —NH group and having one or more heteroatoms selected from the group consisting of N, S, and O.

Specifically, the precursor may include the following (a) to (c):
(a) a compound containing 2 to 6 nitrogen atoms;
(b) a compound containing 2 to 4 nitrogen atoms and one or more oxygen atoms; and
(c) a compound containing one or more nitrogen atoms and one or more sulfur atoms.

For example, the (a) compound may be melamine, dicyandiamide, cyanamide, cyanuric acid, urea, thiourea, or ammonium thiocyanate.

For example, the (b) compound may be cyanuric acid or urea.

For example, the (c) compound may be thiourea or ammonium thiocyanate.

When the precursor includes the (a) to (c) compounds, the supramolecular self-assembly formed via a hydrogen bond, and the like is formed.

In this case, the molar ratio ((a):(c) or (b):(c)) of the (a) or (b) compound to the (c) compound may be in a range of 1:0.2 to 1:2. For example, the molar ratio may be in a range of 1:0.5 to 1:1.5, 1:0.5 to 1:1.3, or 1:0.8 to 1:1.3. When the precursor has the above molar ratio, a supramolecular self-assembly with a uniform hexagonal pillar or hexagonal shape may be formed.

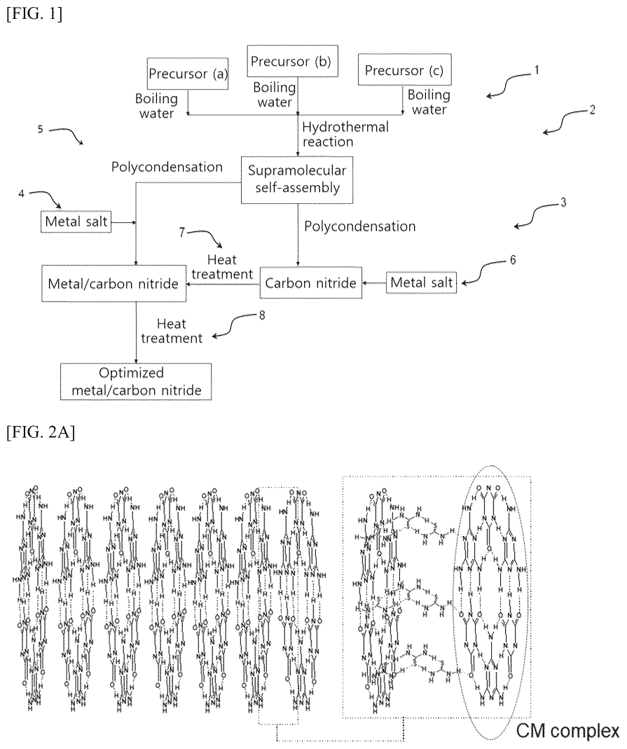

Referring to FIG. 1, each of the precursors may be dissolved in a solvent prior to the hydrothermal reaction (step 1), and the solvent may be water. In this case, the solvent may be used in an amount of 10 mL/g to 30 mL/g. That is, a solution may be formed by dissolving the precursor in water and stirring the resulting mixture at 60° C. to 140° C. for 5 to 30 minutes.

The hydrothermal reaction (corresponding to step 2 in FIG. 1) may be performed at 60° C. to 180° C. or 80° C. to 120° C. for 1 to 12 hours or 4 to 8 hours after each of the solutions is transferred to a reactor. After the hydrothermal reaction, the reaction product may be pulverized, washed, and dried.

Also, the present invention provides a carbon nitride including a heptazine framework. The carbon nitride may be manufactured using the above-described supramolecular self-assembly. Specifically, the carbon nitride may be manufactured by polycondensing and heat-treating the above-described supramolecular self-assembly.

The polycondensation may be performed at 500° C. to 600° C. for 2 hours to 5 hours. Specifically, the polycondensation may be performed at a temperature of 500° C. to 560° C. or 520° C. to 550° C. for 2 hours to 4 hours under an air or nitrogen (N2) atmosphere.

Also, the heat treatment may be performed at 450° C. to 550° C. or 500° C. to 550° C. for 1 hour to 5 hours or 2 hours to 4 hours.

For the carbon nitride, a peak representing C—C binding energy is present at 284.8±1 eV and a peak representing an N—C═N bond is present at 288.1±1 eV, as analyzed by C is X-ray photoelectron spectroscopy (XPS). Here, when it is assumed that the highest peak value observed at 284.8±1 eV is I₁ and the highest peak value observed at 288.1±1 eV is 12, I₂/I may be greater than or equal to 2. For example, I₂/11 may be greater than or equal to 3, greater than or equal to 4, greater than or equal to 5, or greater than or equal to 7.

As the carbon nitride according to the present invention, a condensed carbon nitride having a high N—C═N bond ratio as described above may be provided by subjecting the above-described supramolecular self-assembly to polycondensation and heat treatment under specific conditions.

Also, for the carbon nitride, a peak representing —N—(C)₃ binding energy is present at 398.7±0.1 eV, a peak representing a C—N═C bond is present at 399.1±0.1 eV, and a peak representing —N—H binding energy is present at 401.0±0.1 eV, as analyzed by N is X-ray photoelectron spectroscopy (XPS). Here, when it is assumed that the highest peak value observed at 398.7±0.1 eV is I₃ and the highest peak value observed at 399.1±0.1 eV is I₄, I₄/I₃ may be greater than or equal to 2. For example, I₄/I₃ may be greater than or equal to 2, greater than or equal to 2.5, greater than or equal to 2.8, or greater than or equal to 3.

As the carbon nitride according to the present invention, a condensed carbon nitride having a high C—N═C bond ratio as described above may be provided by subjecting the above-described supramolecular self-assembly to polycondensation and heat treatment under specific conditions.

Also, the carbon nitride may have a bandgap energy of 2.7 eV to 3.0 eV. Such bandgap energy may be attributed to the high N—C═N and C—N═C bond ratios when the above-described supramolecular self-assembly is subjected to polycondensation and heat treatment under specific conditions. When the carbon nitride has such bandgap energy, the light absorption rate for visible light may be excellent.

The carbon nitride may exhibit strong light absorption in a spectrum ranging from 200 to 790 nm when optical properties are measured using diffuse reflectance spectroscopy (DRS).

Also, the present invention provides a photocatalyst including the above-described carbon nitride and a metal oxide formed on the surface of the carbon nitride and/or inside the carbon nitride.

The metal oxide may be a metal oxide including at least one metal selected from tungsten (W), vanadium (V), and molybdenum (Mo).

As the photocatalyst of the present invention, a photocatalyst may be manufactured by dispersing the above-described supramolecular self-assembly in a solvent and adding a metal-containing precursor (corresponding to step 4 in FIG. 1), followed by condensation polymerization (corresponding to step 5 in FIG. 1) and heat treatment (corresponding to step 8 in FIG. 1), or a photocatalyst may be manufactured by polycondensing the above-described supramolecular self-assembly (corresponding to step 3 in FIG. 1), dispersing the supramolecular self-assembly in a solvent, and adding a metal-containing precursor (corresponding to step 6 in FIG. 1), followed by heat treatment (corresponding to step 7 in FIG. 1).

The metal-containing precursor may be a metal salt, for example, ammonium tungstate (IV), ammonium molybdate tetrahydrate or ammonium vanadate (V).

Specifically, when the metal is tungsten, the photocatalyst may be manufactured by dispersing the above-described supramolecular self-assembly in a solvent, and adding a metal-containing precursor, followed by polycondensation and heat treatment. In this case, the metal may be included in an amount of 0.01 to 5 parts by weight of based on 100 parts by weight of the supramolecular self-assembly. When the amount of metal satisfies the above range, the metal may exhibit photocatalytic activity suitable for wastewater disposal under visible light while having a moderate bandgap range. The polycondensation and heat treatment may use the reaction conditions as described above.

Also, when the metal is vanadium or molybdenum, the photocatalyst may be manufactured by polycondensing the above-described supramolecular self-assembly, dispersing the supramolecular self-assembly in a solvent, and adding a metal-containing precursor, followed by heat treatment. In this case, the metal may be included in an amount of 1 to 20 parts by weight based on 100 parts by weight of the polycondensed supramolecular self-assembly. When the amount of the metal satisfies the above range, the metal may exhibit photocatalytic activity suitable for wastewater disposal under visible light while having a moderate bandgap range. The polycondensation may use the reaction conditions as described above, and the heat treatment may be performed at a temperature of 450° C. to 550° C. for a period of 5 to 60 minutes.

The photocatalyst manufactured by the manufacturing method may provide a large number of electron-hole pairs by forming a heterojunction between carbon nitride and a metal or metal oxide. This heterojunction may generate more active species (radicals, and the like) capable of reacting with organic compounds, may broaden the light absorption range, and may increase photocatalytic activity by improving oxidation/reduction reactions.

The photocatalyst may have an average diameter of 5 to 100 nm, for example, an average diameter of 5 to 50 nm, 5 to 30 nm, or 5 to 20 nm. Also, the photocatalyst may have a pore size of 30 nm or more or 30 nm to 80 nm, a pore volume of 0.3 cm³/g or more or 0.3 cm³/g to 0.8 cm³/g, and a BET specific surface area of 100 m²/g or more or 100 m²/g to 150 m²/g.

For the photocatalyst, a peak representing C—C binding energy is present at 284.8±1 eV and a peak representing an N—C═N bond is present at 288.1±1 eV, as analyzed by C is X-ray photoelectron spectroscopy (XPS). Here, when it is assumed that the highest peak value observed at 284.8±1 eV is I₁ and the highest peak value observed at 288.1±1 eV is 12, I₂/I₁ may be greater than or equal to 2. For example, I₂/I₁ may be greater than or equal to 3, greater than or equal to 4, greater than or equal to 5, or greater than or equal to 7.

As the photocatalyst according to the present invention, a photocatalyst having a high C—N═C bond ratio and excellent photocatalytic activity as described above may be provided by subjecting the above-described supramolecular self-assembly and a metal salt to polycondensation and heat treatment under specific conditions to form a heterojunction between the supramolecular self-assembly and the metal salt.

Also, for the photocatalyst, a peak representing —N—(C)₃ binding energy is present at 398.7±0.1 eV, a peak representing a C—N═C bond is present at 399.1±0.1 eV, and a peak representing —N—H binding energy is present at 401.0±0.1 eV, as analyzed by N is X-ray photoelectron spectroscopy (XPS). Here, when it is assumed that the highest peak value observed at 398.7±0.1 eV is 13 and the highest peak value observed at 399.1±0.1 eV is 14, I₄/I₃ may be greater than or equal to 2. For example, I₄/I₃ may be greater than or equal to 2, greater than or equal to 2.5, greater than or equal to 2.8, or greater than or equal to 3.

As the photocatalyst according to the present invention, a photocatalyst having a high C—N═C bond ratio and excellent photocatalytic activity as described above may be provided by subjecting the above-described supramolecular self-assembly and a metal salt to polycondensation and heat treatment under specific conditions to form a heterojunction between the supramolecular self-assembly and the metal salt.

Also, the photocatalyst a bandgap energy of 2.7 eV to 3.0 eV. Such bandgap energy may be attributed to the high N—C═N and C—N═C bond ratios when the above-described supramolecular self-assembly is subjected to polycondensation and heat treatment under specific conditions. When the photocatalyst has such bandgap energy, it may have the light absorption rate for visible light may be excellent.

### MODE FOR INVENTION

Hereinafter, specific embodiments of the present invention are presented. However, it should be understood that the embodiments described below are provided only for illustrating or describing the present invention in detail and are not intended to limit the present invention.

The following examples were conducted at a batch scale using different components to improve photocatalytic activity, and include a characterization method for evaluating the optical, physical and chemical properties. A schematic synthesis method of the photocatalyst is shown in FIG. 1.

Analysis of X-ray diffraction (XRD) spectra was performed by D8 ADVANCE (Bruker) using monochromatic Cu-Kα radiation (λ=1.5418 Å) in a 20 range of 100 to 800 at room temperature.

X-ray photoelectron spectroscopy (XPS) was performed by an ESCALAB 250XI X-ray photoelectron spectrometer (Thermo Fisher Scientific) using a monochromatic Al-Kα source having an energy step size of 1.0 eV under an ultrahigh vacuum of 1.0×10⁻¹⁰ Torr

Transmission electron microscope (TEM) imaging was performed using JEM-2100F (JEOL) at an accelerating voltage of 200 Kv.

Nitrogen adsorption-desorption isotherms were measured using ASAP2020 equipment (Micromeritics Instruments), and all samples were degassed at 150° C. for 3 hours before measurement. The specific surface area and the pore size distribution were extracted using the Brunauer-Emmett-Teller (BET) and Barrett-Joyner-Halenda (BJH) equations, respectively.

Fourier transform infrared (FT-IR) spectra were recorded for samples made from KBR pellets using Model-670/620 (Varian) in a range of 4,000 to 400 cm⁻¹.

Field emission scanning electron microscopy (FE-SEM) was performed by FEI Nova NanoSEM using a Schottky-type thermal FE gun operating at 15 kV.

UV-visible diffuse reflectance spectra (DRS) were measured by a Cary 5000 spectrophotometer (Agilent) using BaSO₄ as a reference. The bandgap was estimated by the Kubelka-Munk theory and a (F(R)hv)ⁿ vs (hv) Tauc plot shown in Equation 1 below.

(1−R_{∞})²/2×R_{∞}=F(R) [Equation 1]
wherein F (R), R_{∞}, h, and v represent a Kubelka-Munk function, layer reflectance, Planck's constant, and a radiation frequency, respectively. The n values were considered to be 0.5 and 2 for direct and indirect allowed transitions of a semiconductor. The bandgap energy (E_{g}) was calculated by extrapolating a linear section of the spectrum to the hv axis.

### Preparation Example and Comparative Examples 1 to 3: Manufacture of Supramolecular Self-Assembly

3.0 g of melamine, 3.0 g of cyanuric acid, and 3.0 g of thiourea were individually added to 60 mL of boiling water, and each was stirred for 30 minutes to obtain a clear solution. Each of the solutions was quickly transferred to a Teflon-lined autoclave reactor and stirred for 30 minutes. The autoclave was sealed and naturally cooled in an oven at 100° C. for 4 hours. Thereafter, the suspension was centrifuged and dried overnight at 80° C. in the oven. The resulting solid was collected, pulverized, and rinsed several times with distilled water to obtain a supramolecular self-assembly sample.

As a precursor, samples were prepared in the same manner as in Preparation Example, except that melamine, cyanuric acid, and thiourea were used alone in Comparative Examples 1 to 3, respectively.

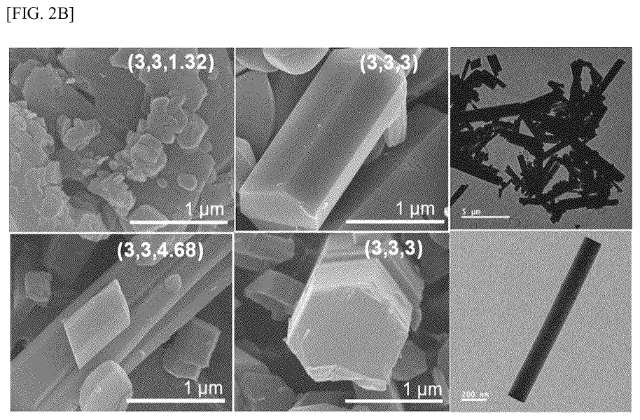

FIG. 2A shows a conceptual diagram of the obtained supramolecular self-assembly, and FIG. 2B shows a scanning electron microscope (SEM) image of the supramolecular self-assembly. Referring to FIG. 2A, it was confirmed that a melamine-cyanurate complex including a heptazine framework and a 1,3,5-triazinane framework is formed in a two-dimensional shape, and a thiourea dimer containing sulfur (S) connects the two-dimensional complexes via a hydrogen bond to form a three-dimensional structure. Referring to FIG. 2B which shows the SEM image of the supramolecular self-assembly according to the molar ratio of the precursors (melamine, cyanuric acid, and thiourea), it can be also seen that the supramolecular self-assembly (3, 3, 3) obtained in Preparation Example has a hexagonal shape with faces. This is similar to the monoclinic space group C2/m. When the molar ratio of thiourea to melamine or cyanuric acid was low (3,3,1.32) or high (3,3,4.68), a hexagonal prism shape was not generated or a uniform supramolecular self-assembly was not formed. These results imply that the presence of sulfur-containing thiourea molecules may provide a regular orientation with respect to other starting materials, thereby forming a new supramolecular self-assembly.

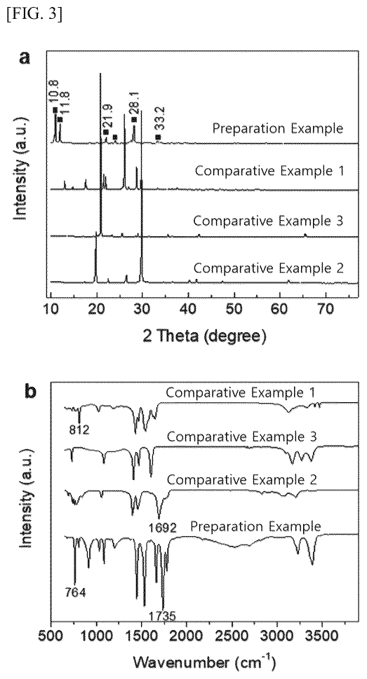

Referring to FIG. 3A, it was confirmed that an X-ray diffraction pattern of the supramolecular self-assembly obtained in Preparation Example 1 shows new peaks at 2θ=10.8°, 11.8°, 21.9°, and 28.1°, indicating that the supramolecular self-assembly has new crystals and structures. In particular, all pivotal diffraction patterns of Comparative Examples 1 to 3 disappeared from the pattern of Preparation Example, indicating that a new supramolecular self-assembly having a new orientation was formed by a combination of the starting materials.

Also, FIG. 3B shows the analysis results of Fourier transform infrared spectroscopy (FT-IR). Fourier transform infrared spectroscopy may provide important information on vibrational coupling in molecular structures. Referring to FIG. 3B, the sample prepared in Preparation Example showed a significant difference in spectrum due to the formation of hydrogen bonds between starting materials compared to those of Comparative Examples 1 to 3. It can be seen that new and prominent peaks appeared in a region of 1,357 to 1,813 cm⁻¹, indicating that new interactions occurred within the supramolecular self-assembly of Preparation Example. Also, it can be seen that the melamine molecule used in Comparative Example 1 and the cyanuric acid molecule used in Comparative Example 2 were connected via hydrogen bonds of N—H . . . O and N—H . . . N in the supramolecular self-assembly of Preparation Example. Specifically, the supramolecular self-assembly manufactured according to Preparation Example of the present invention had a C═O stretching vibration peak shifted from 1,692 to 1,735 cm⁻¹, as compared to Comparative Example 1, and had a triazine ring vibration peak shifted from 810 to 765 cm⁻¹, as compared to Comparative Example 2.

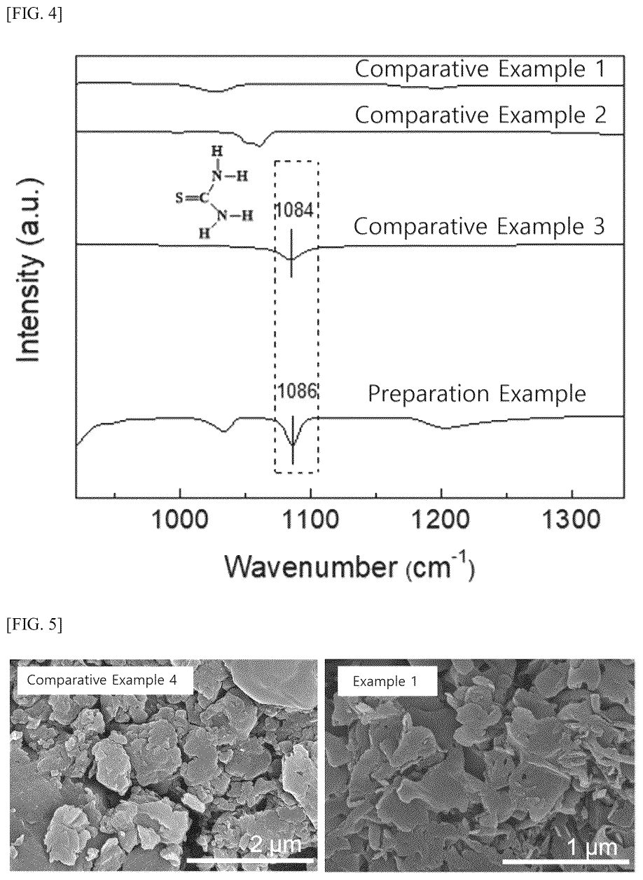

Also, referring to FIG. 4 which shows an enlarged diagram of the resulting FT-IR image corresponding to the sulfur region, the solid state of the molecule in Comparative Example 3 showed a C═S stretching vibration at 1,084 cm⁻¹, but no peaks were observed in this region in the case of Comparative Examples 1 and 2. Also, the supramolecular self-assembly of Preparation Example had a more distinct C═S peak, and the peak shifted to a higher frequency, which could be considered due to non-covalent interactions of other molecules unlike the molecule in Comparative Example 3. Therefore, based on the XRD and FT-IR results, it can be seen that that a new supramolecular self-assembly was formed in Preparation Example through the non-covalent interactions of the starting materials used in Comparative Examples 1 to 3, respectively.

### Example 1 and Comparative Examples 4 and 5: Manufacture of Carbon Nitride

3.0 g of the white solid of Preparation Example was placed in a crucible with a lid, and calcined at 540° C. for 4 hours at a heating rate of 2.5° C./min under a nitrogen atmosphere to obtain a carbon nitride in which the supramolecular self-assembly was condensed. Also, the carbon nitride was further heat-treated at 540° C. for an hour at a heating rate of 2.5° C./min under an air atmosphere to obtain an optimized carbon nitride, which was used as the photocatalyst.

In Comparative Example 4, 3.0 g of melamine was placed in a crucible with a lid, and calcined at 540° C. for 3 hours at a heating rate of 2.5° C./min under an air atmosphere to obtain a carbon nitride. In Comparative Example 5, 3.0 g of thiourea was placed in a crucible with a lid and calcined at 540° C. for 3 hours at a heating rate of 2.5° C./min under an air atmosphere to obtain a carbon nitride.

Referring to FIG. 5, it can be seen from the scanning electron microscope (SEM) image that the photocatalyst manufactured in Example 1 exhibited nanosheets with a regular shape. Through comparison of Example 1 with Comparative Example 4, it can be seen that significant improvement in properties is possible when the new supramolecular self-assembly was used to manufacture a carbon nitride photocatalyst.

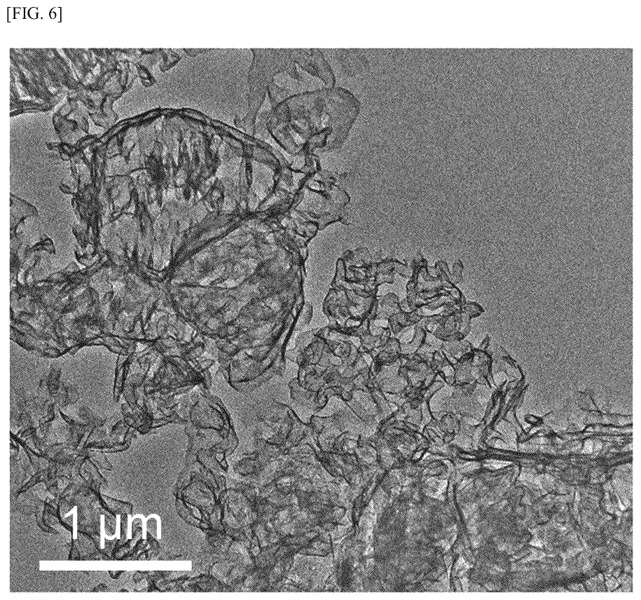

Referring to FIG. 6, the transmission electron microscopy (TEM) image of the photocatalyst of Example 1 is fully compatible with the SEM results. Two-dimensional sheet-type nanostructures may provide excellent conditions for the transition of electron-hole pairs on the surface of the photocatalyst and may serve as a substrate for metal oxide nanoparticles.

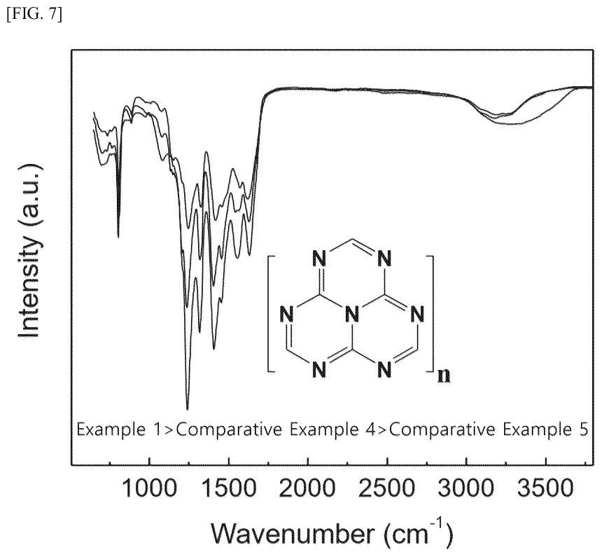

Referring to FIG. 7, it can be seen through the FT-IR spectrum that a conjugated system of heptazine units was deformed in a thermal polycondensation process because the peak area of the triazine ring in a stretching mode increased significantly from 1,200 to 1,650 cm⁻¹ in the case of the photocatalyst manufactured in Example 1. Therefore, the thermal polymerization of the supramolecular self-assembly of Preparation Example prevents the sublimation of monomers and intermediates during heating to provide a suitable environment for the formation of carbon nitride.

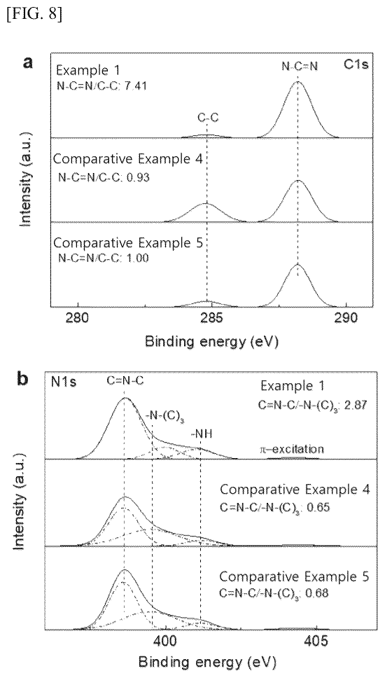

Also, an effect of the supramolecular self-assembly on the final photocatalyst was further confirmed using X-ray photoelectron spectroscopy (XPS) spectrum analysis. FIG. 8A shows high-resolution C 1s XPS results for the photocatalysts prepared in Comparative Example 4, Comparative Example 5, and Example 1. The high-resolution C is spectrum of the photocatalyst prepared in Example 1 shows two typical peaks located at 284.8 and 288.1 eV, which are related to C—C and NC═N groups, respectively.

The ratios of NC═N to C—C in the photocatalysts of Example 1 and Comparative Examples 4 and 5 were 7.41, 1.0, and 0.93, respectively. These results suggest that more heptazine units were generated through modified thermal polycondensation of the new starting material manufactured in Preparation Example.

Referring to FIG. 8B, the N1 spectrum shows three peaks at 398.7, 399.7, and 401.0 eV, which correspond to C═N—C, —N—(C)₃, and —N—H groups, respectively. In this case, the ratios of C═N—C to N—(C)₃ in the samples manufactured in Example 1 and Comparative Examples 4 and 5 were 2.87, 0.65, and 0.68, respectively. The ratio of N—C═N to C—C and the ratio of C═N—C to N—(C)₃ are shown in Table 1.

TABLE 1

| | N—C—N/ | C—N—C/ | Bandgap |
|---|---|---|---|
| Classification | C—C | N—(C)₃ | energy [eV] |
| Example 1 | 7.41 | 2.87 | 2.83 |
| Comparative Example 4 | 1.0 | 0.65 | 2.69 |
| Comparative Example 5 | 0.93 | 0.68 | 2.61 |

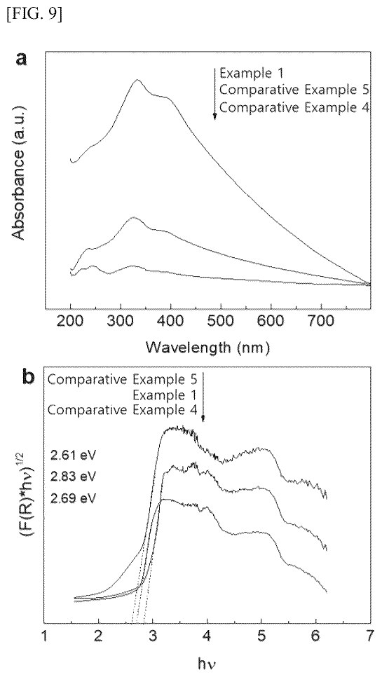

The optical properties of the photocatalyst according to the present invention were evaluated using diffuse reflectance spectroscopy (DRS). Referring to FIG. 9A, the photocatalyst manufactured in Example 1 showed the strongest light absorption across the entire spectrum ranging from 200 to 790 nm compared to Comparative Examples 4 and 5. This is due to better polycondensation process conditions and more heptazine block units determined by FT-IR. Referring to FIG. 9B based on the Kubelka-Munk theory, the bandgap energy of the photocatalyst manufactured in Example 1 is higher than those of the photocatalysts manufactured in Comparative Examples 4 and 5. This blue shift is fully consistent with the SEM and TEM results, in which the particle size and stacked layers were significantly reduced, respectively. The bandgaps of the photocatalysts of Example 1 and Comparative Examples 4 and 5 are shown in Table 1 above. These results indicate that the photocatalyst of the present invention may be used in systems that irradiate indoor visible light because the photocatalyst has excellent optical properties across the entire spectrum.

### Example 2 and Comparative Examples 6 and 7: Metal Oxide-Added Photocatalyst

2.5 g of the supramolecular self-assembly powder manufactured in Preparation Example was dispersed in 100 mL of water by intense sonication for 10 minutes, and then maintained for an hour in an ultrasonic treatment bath. The initial amount of the supramolecular self-assembly has a crucial impact on the final product. Thereafter, 0.2 g of ammonium tungstate (IV) was added as a metal source to a suspension of the supramolecular self-assembly, and vigorously stirred for 15 minutes. Then, the mixture was strongly sonicated for 10 minutes and placed in an ultrasonic water bath in order to obtain metal ions dispersed well in the supramolecular self-assembly particles. The mixture was stirred overnight at room temperature to stabilize the metal ions adsorbed on the surface of the supramolecular self-assembly. The product was dried at 80° C. for 24 hours, and ground in a mortar for uniformity. Then, the ground solid was placed in a crucible with a lid, and polycondensed by tempering at 540° C. for 3 hours at a heating rate of 3.0° C./min. Under an air atmosphere, the metal ions and the supramolecular self-assembly were gradually converted into a heterojunction form into which the metal ions were inserted. The product was washed and centrifuged to dissolve unreacted ions, and dried in air overnight at a temperature of 80° C. To obtain an optimized photocatalyst, the product was also further heat-treated at 540° C. for an hour at a heating rate of 2.5° C./min in air to manufacture the photocatalyst of Example 2 having the final product W.

Also, in Comparative Example 6, 3.0 g of the supramolecular self-assembly was placed in a crucible with a lid, and calcined at 540° C. for 3 hours in air at a heating rate of 2.5° C./min to obtain a carbon nitride photocatalyst. A process of calcining WS₂ at 540° C. for 4 hours to obtain WO₃ was included in Comparative Example 7 to manufacture a photocatalyst.

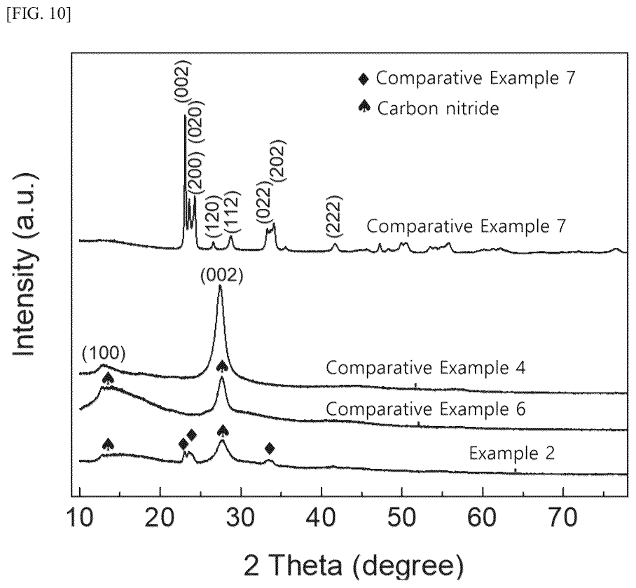

The XRD diffraction patterns of the photocatalysts prepared in Example 2 and Comparative Examples 4, 6, and 7 are shown in FIG. 10. Referring to FIG. 10, the typical carbon nitride manufactured in Comparative Example 4 shows one intense peak at 27.4° (002) and one weak peak at 13.1 (100), which belong to the interlayer stacking and in-plane packing motifs of the heptazine units, respectively. The significant reduction in (002) plane for the photocatalyst manufactured in Example 2 occurs due to the reduced stacking along the c-axis and the deviation from the bulk structure. Also, these physical structural changes may affect nitrogen pots, as evidenced by new deviations from the (100) plane. XRD peaks of WO₃ nanoparticles having (002), (020), and (022) planes appeared in the photocatalyst manufactured in Example 2, which indicates that the tungsten nanoparticles and the supramolecular self-assembly were successfully grown and the supramolecular self-assembly closely interacted with the tungsten nanoparticles. As a result, the interfacial contact between WO₃ and carbon nitride was ensured, and more opportunities for accelerated charge transfer were provided.

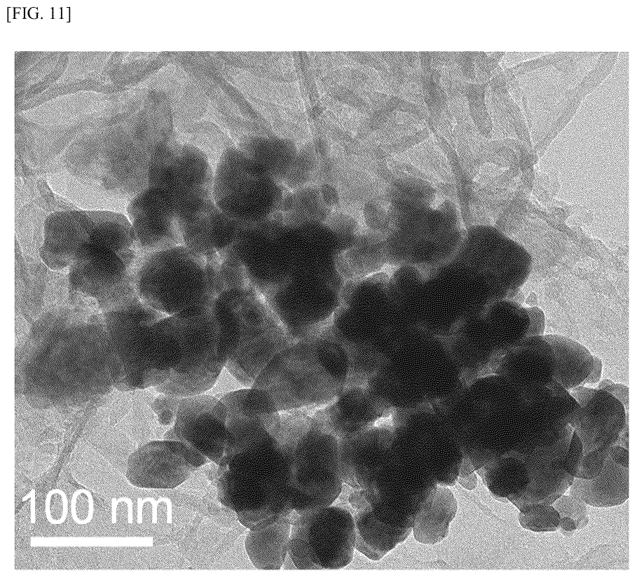

Also, as can be seen from the weak XRD patterns, tungsten ions are prevented from growing excessively on the surface of the carbon nitride because the supramolecular self-assembly may have a confinement effect on the tungsten ions. FIG. 11, which shows a low-magnification TEM image of the photocatalyst manufactured in Example 2, shows small-sized nanoparticles of WO₃ composed of nanocrystals of 20 nm or less attached to ultra-thin carbon nitride nanosheets.

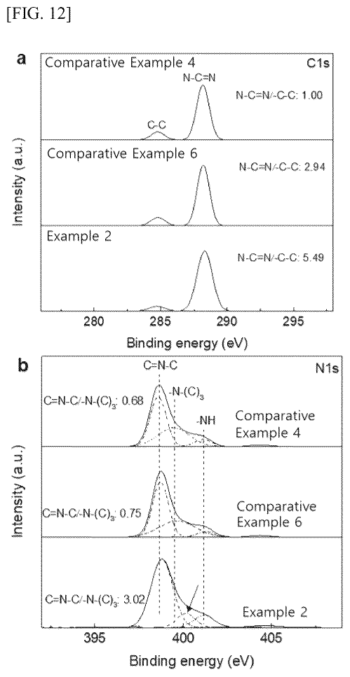

The high-resolution XPS spectrum of C1 in FIG. 12A shows two peaks at binding energies of 284.8 and 288.1 eV, which correspond to C—C and N—C═N bonds, respectively. As shown in FIG. 12A and Table 2, the ratio of N—C═N to C—C in Example 2 increased from 1.0 to 5.49, as compared to Comparative Example 4.

The high-resolution XPS spectrum of N1 for the photocatalyst shows peaks at 398.6, 399.6, and 401.2 eV related to the C—N═C, —N—(C)₃, and —N—H groups, respectively. As shown in FIG. 12B, the ratio of C—N═C to N—(C)₃ increases in the order of Comparative Example 4 (0.68), Comparative Example 6 (0.75), and Example 2 (3.02). This increase suggests more condensation of heptazine units and implies an improvement in the polycondensation process. The XPS peaks related to C═N—C and N—(C)₃ of the photocatalyst manufactured in Example 2 show a slight blue shift compared to Comparative Example 4. In particular, the N—(C)₃ position dramatically shifts to a higher position. The binding energy representing electron density decreases around the N—(C)₃ group. These results are obtained as the ratio of C—N═C to N—(C)₃ increases further and the heptazine ring becomes more condensed. More nitrogen atoms around the N—(C)₃ site can draw more electrons from N—(C)₃, resulting in a positive motion in XPS. The photocatalyst manufactured in Example 2 exhibits an excellent configuration of highly condensed heptazine units that exhibit efficient visible light harvesting.

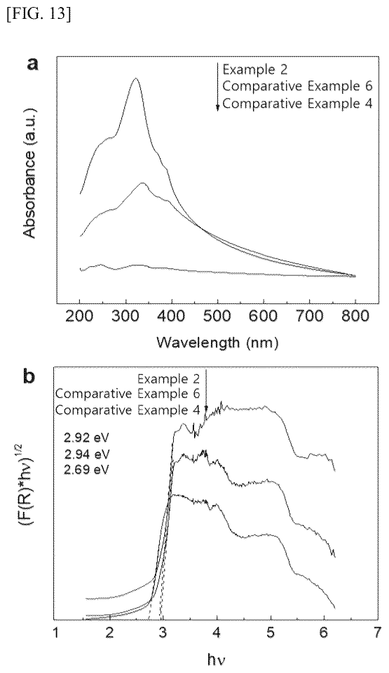

The optical properties and bandgaps of the photocatalysts manufactured in Example 2 and Comparative Examples 4 and 6 were determined by DRS measurement and Kubelka-Munk plotting, as shown in FIGS. 13A and 13B. The photocatalysts manufactured in Example 3 and Comparative Example 6 had excellent absorbance observed across the entire spectrum ranging from 200 to 750 nm compared to the photocatalyst manufactured in Comparative Example 4. This may be because multiple reflections are provided within the structure due to the high surface area and pore volume.

Meanwhile, as confirmed by the XPS results, more heptazine blocks indicated more absorbance antennas and electronic transitions, resulting in improved absorbance ability of the photocatalyst manufactured in Example 2. In particular, although the color of the powder changed from yellow to white and the bandgap increased to 2.94 eV, the optical power did not decrease. The bandgaps of the photocatalysts of Example 2 and Comparative Examples 4 and 6 are shown in Table 2 below.

TABLE 2

| | N—C—N/ | C—N═C/ | Bandgap |
|---|---|---|---|
| Classification | C—C | N—(C)₃ | energy [eV] |
| Example 2 | 5.49 | 3.02 | 2.92 |
| Comparative Example 6 | 2.94 | 0.75 | 2.94 |
| Comparative Example 4 | 1.0 | 0.68 | 2.69 |

For the photocatalyst manufactured in Example 2, further light absorption enhancement was observed in a region of 200 to 450 nm along with a red shift of the bandgap (2.92 eV). This may be due to the tight interface formation established between W03 nanoparticles and carbon nitride. Table 3 below shows the results of nitrogen adsorption-desorption analysis to further investigate the properties of the photocatalysts manufactured in Example 2 and Comparative Examples 4 and 6.

TABLE 3

| | Pore size | Pore volume | BET specific |
|---|---|---|---|
| Classification | [nm] | [cm³/g] | surface area [m²/g] |
| Example 2 | 32.5 | 0.61 | 132 |
| Comparative Example 6 | 20.3 | 0.26 | 90 |
| Comparative Example 4 | 28.15 | 0.045 | 9 |

The surface areas of the photocatalysts manufactured in Example 2 and Comparative Examples 4 and 6 were 132, 90.7, and 9 m²/g, respectively, and the pore volumes were 0.61, 0.26, and 0.045 cm³/g, respectively. Such a significant improvement in pores and surface area of Example 2 was obtained using the new starting material manufactured in Preparation Example by an in-situ process capable of improving visible light absorption and charge mobility.

### Example 3: Metal Oxide-Added Photocatalyst

Mixture A was prepared by dispersing 0.8 g of the carbon nitride manufactured in Example 2 without heat treatment by intense sonication in 100 mL of water for 10 minutes and then maintaining the resulting dispersion for an hour in an ultrasonic treatment bath. The initial amount of the non-heat-treated carbon nitride of Example 2 has a significant impact on the final product. Thereafter, 0.2 g of ammonium vanadate (V) was added as a metal source to Mixture A, and vigorously stirred for 15 minutes. Then, the mixture was strongly sonicated for 10 minutes, and taken out of the ultrasonic treatment bath to obtain a carbon nitride in which metal ions were dispersed well without heat treatment. The mixture was stirred overnight at room temperature to stabilize the metal ions absorbed on the surface of the carbon nitride. The final solid was centrifuged, collected, and then thoroughly rinsed several times with distilled water. The powder was dried at 80° C. for 24 hours, and ground in a mortar for uniformity. Then, the solid was placed in a crucible with a lid, and polycondensed by tempering at 540° C. for 3 hours at a heating rate of 2.5° C./min. Metal ions were converted in air into heterojunction photocatalysts. To obtain an optimized photocatalyst, additional heat treatment was also performed at 540° C. for 30 minutes at a heating rate of 2.5° C./min in air to obtain the final photocatalyst of Example 3.

### Example 4: Metal Oxide-Added Photocatalyst

Mixture B was prepared by dispersing 0.8 g of the carbon nitride manufactured in Example 2 without heat treatment by intense sonication in 100 mL of water for 10 minutes and then maintaining the resulting dispersion for an hour in an ultrasonic treatment bath. The initial amount of the non-heat-treated carbon nitride of Example 2 has a significant impact on the final product. Thereafter, 0.2 g of ammonium molybdate tetrahydrate was added as a metal source to Mixture B, and vigorously stirred for 15 minutes. Then, the mixture was strongly sonicated for 10 minutes, and taken out of the ultrasonic treatment bath to obtain a carbon nitride in which metal ions were dispersed well without heat treatment. The mixture was stirred overnight at room temperature to stabilize the metal ions adsorbed on the surface of the carbon nitride. The final solid was centrifuged, collected, and then thoroughly rinsed several times with distilled water. The powder was dried at 80° C. for 24 hours, and ground in a mortar for uniformity. Then, the solid was placed in a crucible with a lid, and polycondensed by tempering at 500° C. for 3 hours at a heating rate of 2.5° C./min. Metal ions were converted in air into heterojunction photocatalysts. To obtain an optimized photocatalyst, additional heat treatment was also performed at 500° C. for 30 minutes at a heating rate of 2.5° C./min in air to obtain the final photocatalyst of Example 4.

### Experimental Example 1

To evaluate the photocatalytic activity of the present invention including a carbon nitride-based photocatalyst, the following photodegradation experiment was performed.

First, the performance of the photocatalyst was confirmed through photocatalytic degradation of an organic dye such as rhodamine B, which is a typical reference material. Also, the performance of the photocatalyst as a wastewater disposal agent was confirmed by photodegradation of tetracycline, which is not sensitive to visible light irradiation.

A method of measuring an organic compound is as follows.

2.5 mL of a sample was taken, and the concentration was measured using a UV-vis spectrophotometer. Rhodamine B has one prominent absorption peak at λₘₐₓ=553 nm, while tetracycline has two distinct peaks at λₘₐₓ=273 and 356 nm. Photoactivity was calculated using η=(C₀−Cₜ)/C₀. Here, η, C₀, and Cₜ are a photocatalyst efficiency, an initial concentration before light irradiation, and a concentration after light irradiation, respectively.

In another embodiment, the degradation results fit well with a first-order reaction kinetics model according to InC₀/Cₜ=Kₐₚₚt. Here, Kₐₚₚ is an apparent reaction rate constant, and t is an irradiation time.

10 mg of each of the photocatalysts manufactured in Examples 1 to 4 and Comparative Examples 4 and 5 was dispersed at a concentration of 12 mg/L in an aqueous solution (15 to 20 mL) containing rhodamine B as an organic compound. Before irradiation, a Pyrex vial was kept for 60 minutes under dark conditions to reach the adsorption-desorption equilibrium. The vial was placed 10 cm from a 300 W Xe lamp with a 400 nm cut-off filter. Photodegradation efficiency was calculated by centrifuging the photocatalyst for a given time and measuring the concentration of the rhodamine B in the resulting supernatant.

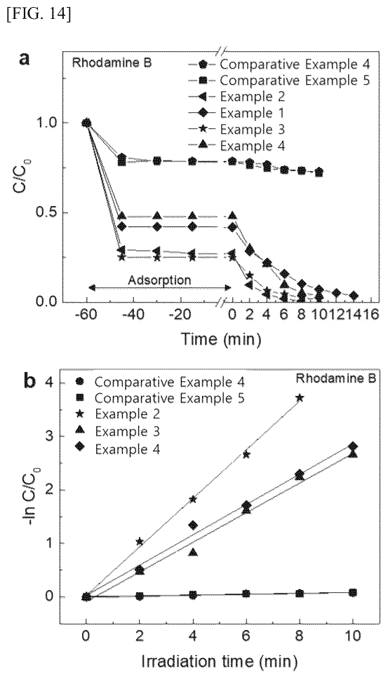

Referring to FIG. 14A, all the main photocatalysts manufactured in Examples 1 to 4 showed photoactivity for degradation of rhodamine B in a very short period of time under visible light irradiation compared to Comparative Examples. For example, the photocatalyst manufactured in Example 1 removed more than 98% of rhodamine B in 14 minutes, while Comparative Examples 4 and 5 showed removal rates of approximately 29% and 36%, respectively. More specifically, the photocatalysts manufactured in Examples 2 and 3 showed a removal rate of 99% or more in 8 minutes when the organic compound was rhodamine B. In particular, the photocatalysts manufactured in Examples 2 and 3 showed an excellent adsorption capacity to adsorb more than 73% of rhodamine B in 15 minutes under dark conditions. Based on the degradation results, the photocatalyst manufactured according to the present invention has excellent optical properties capable of adsorbing a high concentration of an organic compound and degrading the organic compound in a short time under visible light irradiation.

Referring to Table 4 below and FIG. 14B, the photodegradation reaction rate constants of the photocatalysts manufactured in Examples 2, 3, and 4 under visible light irradiation of rhodamine B were 0.453, 0.283, and 0.276 min⁻¹, respectively, which were approximately 57.34, 35.82, and 34.9 times higher than that of the photocatalyst of Comparative Example 5. This performance corresponds to the highest value reported for rhodamine B. Also, the comparative kinetic data indicates that the carbon nitride-based photocatalysts may form electron-hole pairs and significantly reduce the recombination of charge carriers, thereby providing very good photocatalytic efficiency.

TABLE 4

| Classification | R-Square | Kₐₚₚ |
|---|---|---|
| Example 4 | 0.986 | 0.276 |
| Example 3 | 0.990 | 0.283 |
| Example 2 | 0.996 | 0.453 |
| Comparative Example 5 | 0.948 | 0.0079 |
| Comparative Example 4 | 0.910 | 0.0087 |

### Experimental Example 2

According to the characterization results of the photocatalysts according to Examples 1 to 4, the photocatalysts had highly condensed heptazine units and exhibited efficient charge separation and extended light absorption across the entire spectrum. Some experiments were performed using different wavelengths in order to further evaluate the optical properties of the photocatalyst. A method of removing organic compounds involves dispersing the photocatalyst (10 mg), for example, the photocatalyst manufactured in Example 2, into a reaction vial (15 to 20 mL) containing rhodamine B at a concentration of 12 mg/L as the organic compound. The reaction vial was positioned 10 cm from a 300 W Xe lamp as a light source. Before irradiation, a Pyrex vial was kept for 60 min under dark conditions to reach the absorption-desorption equilibrium. The photocatalyst was removed by centrifugation for a given time, and the concentration of rhodamine B in the resulting supernatant was measured at a wavelength of 553 nm using a UV-vis spectrophotometer. In this case, the visible light region was adjusted variously using various cut-off filters such as 400, 420, 435, 495, and 550 nm filters.

Referring to Table 5 below, it is clearly shown that no band pass filter may prevent the photodegradation of rhodamine by the photocatalyst manufactured in Example 2. Based on these results, it was clearly confirmed that there was an exact relationship between the optical and organization characterization results and performance under visible light irradiation. More specifically, the photocatalyst manufactured in Example 2 degraded rhodamine B in a short time of 15 minutes under irradiation with visible light having a wavelength of 550 nm or more. Therefore, the photocatalyst according to the present invention had extended light absorption, efficient charge separation, and impressive photocatalytic performance.

TABLE 5

| Band pass filter | Photodegradation rate of | Time |
|---|---|---|
| (nm) | rhodamine B of Example 2 | (min) |
| 400 | Approximately 100% | 8 |
| 420 | Approximately 100% | 8 |
| 435 | Approximately 100% | 8 |
| 495 | Approximately 100% | 8 |
| 550 | Approximately 100% | 15 |

### Experimental Example 3

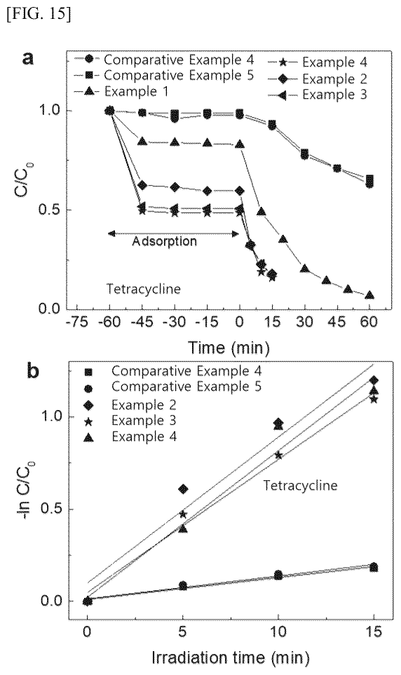

To evaluate the photocatalytic performance of the photocatalyst according to the present invention, the photocatalysts manufactured in Examples 1 to 4 and Comparative Examples 4 and 5 were used as a wastewater disposal agent to remove tetracycline. In this case, 10 mg of the photocatalyst was dispersed in an aqueous solution (15 to 20 mL) containing tetracycline at a concentration of 20 mg/L as an organic compound. Before irradiation, a Pyrex vial was kept for 60 min under dark conditions to reach the absorption-desorption equilibrium. The vial was positioned 10 cm from a 300 W Xe lamp with a 400 nm cut-off filter. The photocatalyst was centrifuged for a given time, and the concentration of tetracycline of the resulting supernatant was measured to calculate photodegradation efficiency. Referring to FIG. 15A, all the photocatalysts manufactured in Examples 1 to 4 degraded tetracycline in a very short period of time under visible light irradiation compared to Comparative Examples. For example, the photocatalyst manufactured in Example 1 removed more than 92% of tetracycline in 60 minutes, while both Comparative Examples 4 and 5 showed a removal rate of approximately 32% or less. More specifically, the photocatalysts manufactured in Examples 2 to 4 showed a removal rate of more than 82% in 15 minutes when the organic compound was tetracycline. The photocatalysts manufactured in Examples 2 and 3 had an excellent tetracycline adsorption capacity, and thus showed an adsorption capacity of more than 50% in 15 minutes under dark conditions. Based on the degradation results, the photocatalyst manufactured according to the present invention has excellent optical properties capable of adsorbing a high concentration of an organic compound and degrading the organic compound in a short time under visible light irradiation.

Referring to Table 6 below and FIG. 15B, the tetracycline photodegradation reaction rate constants of the photocatalysts manufactured in Examples 2, 3, and 4 under visible light irradiation were 0.079, 0.072, and 0.078 min⁻¹, respectively, which were approximately 6.7, 6.6, and 6.1 times higher than that of the photocatalyst of Comparative Example 4. This performance corresponds to the highest value reported for tetracycline. This is due to the close interface between metal oxide nanoparticles and carbon nitride nanosheets, showing strong absorption capacity across the entire spectrum range (200 to 700 nm).

TABLE 6

| Classification | R-Square | Kₐₚₚ |
|---|---|---|
| Example 4 | 0.955 | 0.0785 |
| Example 3 | 0.981 | 0.0721 |
| Example 2 | 0.933 | 0.0791 |
| Comparative Example 5 | 0.958 | 0.0124 |
| Comparative Example 4 | 0.971 | 0.0118 |

### Experimental Example 4

To closely analyze the performance of the photocatalyst manufactured according to the present invention, intermediates in tetracycline photodegradation were identified using liquid chromatography-mass spectrometry (LC-MS). The same method as described in Experimental Example 3 was used to prepare samples for LC-MS analysis. For example, 10 mg of the photocatalyst manufactured in Example 2 was dispersed in an aqueous solution (15 to 20 mL) containing tetracycline at a concentration of 12 mg/L as an organic compound. Before irradiation, a Pyrex vial was kept for 60 minutes under dark conditions to reach the absorption-desorption equilibrium. Two samples were extracted under dark conditions at reaction times of 15 and 60 minutes, named “Adsorption/15 min” and “Adsorption/60 min”, along with a standard solution (STD) of tetracycline for LC-MS analysis. Photodegradation of tetracycline was performed using a 300 W Xe lamp with a 400 nm cut-off filter. The reaction solution was sampled for reaction times of 15 and 30 minutes under visible light irradiation.

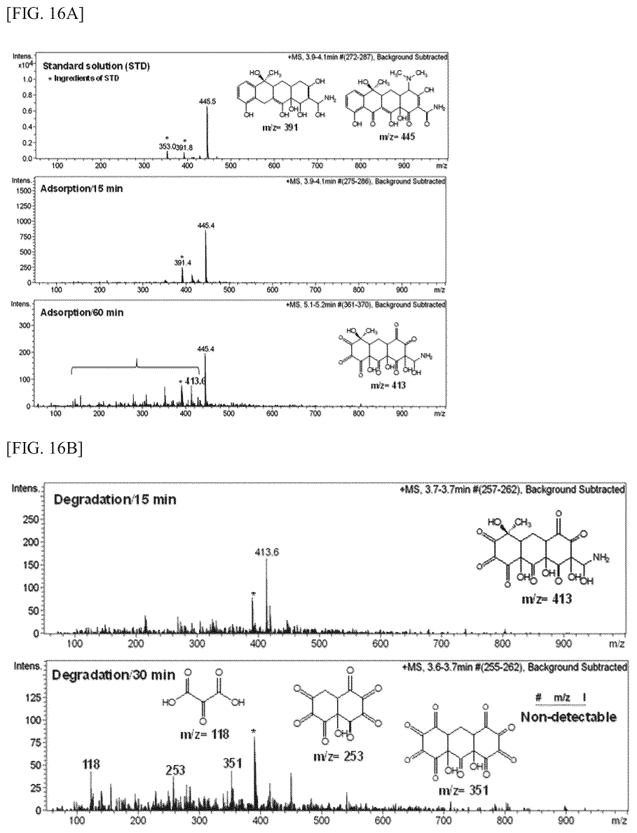

Referring to FIGS. 16A and 16B, the STD solution showed one peak at m/z=445.5 and two peaks with high intensity at m/z=353.0 and m/z=391.8. The former corresponds to the characteristic peak of tetracycline, while the other peaks are components of STD (indicated by an asterisk). After 15 minutes under dark conditions, the intensity of m/z=445.5 significantly decreased from 10,000 to 1,500, which was completely consistent with the tetracycline absorbance by the surface of the photocatalyst. In particular, the decreasing trend of m/z=445.5 with several intermediates extended the dark state to 60 minutes. The main intermediate appears at m/z=413.6, which results from the fact that the ·O₂⁻and ·OH radicals attack the tetracycline molecules, as shown in the inset of FIG. 16. This suggests that the photocatalyst manufactured in Example 2 may generate active species even without light irradiation. This may be due to the strong capability of WO₃ nanoparticles as an oxidation catalyst and the combination of WO₃ nanoparticles and carbon nitride nanosheets. These results could be confirmed by the spectrum results examined for 15 minutes in which only the peak at m/z=413.6 is clearly observed without any presence of peaks corresponding to tetracycline. By maintaining the irradiation up to 30 minutes, some non-detectable intermediates with low intensity appear around m/z=118, m/z=253, and m/z=351, the corresponding structures of which are shown in the inset of FIG. 16. These intermediates generated in a short time after irradiation clearly exhibited four important phenomena: (i) amide group oxidation, (ii) double bond oxidation, (iii) dealkylation, and (iv) ring opening. As a result, the photocatalyst according to the present invention could completely remove a high concentration of the organic compounds in water.

### Experimental Example 5

The photocatalyst manufactured according to the present invention exhibited impressive photocatalytic activity across the entire spectrum, and thus the performance of the photocatalyst was further evaluated under indoor lighting such as in building, laboratory, and office lighting.

This experimental method involves removing organic compounds such as rhodamine B and tetracycline from water under indirect indoor lighting. To evaluate photocatalytic activity, for example, 10 mg of the photocatalyst manufactured in Example 2 was added to an aqueous solution (15 to 20 mL) containing tetracycline or rhodamine at a concentration of 10 mg/L as the organic compound. A Pyrex vial was stirred for 60 minutes under dark conditions to reach the absorption-desorption equilibrium. The reaction vial was placed under indoor light illumination provided by a 32 W Osram linear fluorescent lamp suspended from the ceiling. In this case, the external light in the indoor system may be less than expected because the Pyrex wall of the reaction vial may block or absorb ultraviolet rays from the outside. The photocatalyst was centrifuged for a given time, and the concentration of tetracycline in the resulting supernatant was measured to calculate the photodegradation efficiency.

Referring to Table 7 below, the photodegradation of rhodamine B in the photocatalyst manufactured in Example 2 under an indoor lighting system reached more than 96% after 20 hours. On the other hand, no significant degradation was observed in the photocatalyst manufactured in Comparative Example 4 or 5. In the photocatalyst manufactured in Example 2, 73% or more of tetracycline was also degraded after 20 hours under indoor lighting, but no obvious degradation was observed in the photocatalyst manufactured in Comparative Example 4 or 5.

TABLE 7

| | Photodegradation | Photodegradation | Time |
|---|---|---|---|
| Indoor lighting | rate of rhodamine B | rate of tetracycline | (h) |
| Example 2 | 96.3% | 77.2% | 20 |
| Comparative Example 5 | 22.1% | 10.9% | 20 |

From these results, it can be seen that the present invention proposes a simple, scalable, and more efficient method to manufacture a carbon nitride-based photocatalyst containing highly condensed carbon nitride nanosheets and well-distributed metal oxide nanoparticles. This method utilizes a novel supramolecular self-assembly and includes a thermal condensation process. This induces a modified solid reaction, and enhances dispersibility with limited growth of metal oxide nanoparticles, thereby forming a tight interface between the metal oxide and carbon nitride. The photocatalyst according to the present invention exhibited extended light absorption across the entire spectrum, excellent charge separation, and impressive performance even under dark conditions.

### INDUSTRIAL APPLICABILITY

The visible light-based photocatalyst according to the present invention has high photocatalytic activity for photodegradation of organic contaminants, and thus may be applied to wastewater disposal. Also, the photocatalyst of the present invention may be used in a slurry form in indoor lighting systems. Therefore, the nanocomposite of the present invention facilitates industrial application in water and wastewater disposal, and the visible light-based nanocomposite may also be used in air filtration systems.

1. A supramolecular self-assembly comprising:
a plurality of complex units formed by hydrogen bonding of two or more nitrogen-containing compounds to each other; and
a linker unit configured to connect the plurality of complex units via a hydrogen bond,
wherein the nitrogen-containing compounds and the linker unit each independently include a —NH group and one or more heteroatoms capable of hydrogen bonding with the —NH group and selected from the group consisting of N, S, and O.

2. The supramolecular self-assembly of claim 1, wherein at least one of the nitrogen-containing compounds contains S or O, which is different from the heteroatoms included in the linker unit.

3. The supramolecular self-assembly of claim 1, wherein the nitrogen-containing compounds include a first nitrogen-containing compound having an —NH group and N, and a second nitrogen-containing compound having an —NH group and O, and
the linker includes a compound containing an —NH group and S.

4. The supramolecular self-assembly of claim 1, wherein the plurality of complex units include a 1,3,5-triazine framework and a 1,3,5-triazinane framework.

5. The supramolecular self-assembly of claim 1, wherein the linker includes thiourea, a thiourea dimer, or a combination thereof.

6. The supramolecular self-assembly of claim 1, which shows a peak at 2θ=10.8°±0.4°, 11.8°±0.4°, 28.1°±0.4°, or 33.2°±0.4°, as measured by X-ray diffraction using CuKα rays.

7. The supramolecular self-assembly of claim 1, which shows a peak at 1084±20 cm⁻¹, as measured by FT-IR.

8. A method of manufacturing a supramolecular self-assembly, the supramolecular self-assembly being manufactured by a hydrothermal reaction using a precursor,
wherein the precursor includes,
a nitrogen-containing compound having an —NH group; and
a compound capable of hydrogen bonding with the —NH group and having one or more heteroatoms selected from the group consisting of N, S, and O.

9. The method of claim 8, wherein the precursor includes the following (a) to (c):
(a) a compound containing 2 to 6 nitrogen atoms;
(b) a compound containing 2 to 4 nitrogen atoms and one or more oxygen atoms; and
(c) a compound containing one or more nitrogen atoms and one or more sulfur atoms.

10. The method of claim 9, wherein the molar ratio of the (a) or (b) compound to the (c) compound is in a range of 1:0.2 to 1:2.

11. The method of claim 8, wherein the hydrothermal reaction is performed at 60° C. to 180° C. for 1 to 12 hours after the precursor is dissolved in a solvent.

12. A carbon nitride comprising a heptazine framework,
wherein a peak representing C—C binding energy is present at 284.8±1 eV and a peak representing an N—C═N bond is present at 288.1±1 eV, as analyzed by C 1s X-ray photoelectron spectroscopy (XPS), and when it is assumed that the highest peak value observed at 284.8±1 eV is I₁, and the highest peak value observed at 288.1±1 eV is I₂, I₂/I₁ is greater than or equal to 2.

13. The carbon nitride of claim 12, which has a bandgap energy of 2.7 eV to 3.0 eV.

14. A method of manufacturing a carbon nitride, comprising:
polycondensing and heat-treating the supramolecular self-assembly of claim 1 to manufacture a carbon nitride.

15. The method of claim 14, wherein the polycondensation is performed at 500° C. to 600° C. for 2 to 5 hours.

16. The method of claim 14, wherein the heat treatment is performed at 450° C. to 550° C. for 1 to 5 hours.

17. A photocatalyst comprising:
the carbon nitride of claim 12; and
a metal oxide formed on a surface of the carbon nitride and/or inside the carbon nitride.

18. The photocatalyst of claim 17, wherein the metal oxide includes at least one metal oxide selected from tungsten, vanadium, and molybdenum.

19. The photocatalyst of claim 17, which has a pore size of 30 nm or more, a pore volume of 0.3 cm³/g or more, and a BET specific surface area of 100 m²/g or more.

20. A method of manufacturing a photocatalyst, comprising:
polycondensing a supramolecular self-assembly; and
heat-treating the polycondensed self-assembly,
wherein the polycondensing is performed by dispersing a metal-containing precursor and the self-assembly in a solvent to perform the polycondensation, or
the heat-treating is performed by dispersing a metal-containing precursor and the polycondensed self-assembly in a solvent to perform the heat treatment.
